(19) United States
(12) Patent Application Publication
(10) Pub. No.: US 2012/0089104 A1
(43) Pub. Date: Apr. 12, 2012

(54) ANTIVIRAL TRANSDERMAL PATCH AND METHOD FOR PRODUCING THE SAME

(76) Inventors: Yefu WANG, Wuhan (CN); Wei LI, Wuhan (CN); Xiaozhou ZHOU, Wuhan (CN)

(21) Appl. No.: 13/108,967

(22) Filed: May 16, 2011

Related U.S. Application Data
(63) Continuation of application No. PCT/CN2010/077636, filed on Oct. 12, 2010.

(51) Int. Cl. A61M 35/00; B05D 3/00; B05D 5/00; B05D 1/38

(52) U.S. Cl. 604/290; 604/304; 604/307; 427/2.31

(57) ABSTRACT

An antiviral transdermal patch including a backing layer, a viscous polymer layer, and a protection film layer. The viscous polymer layer includes an antiviral agent, a viscous polymer, and a transdermal enhancer. The transdermal enhancer is laurocapram or a mixture thereof. The antiviral agent is a nucleoside antiviral drug with a daily delivery rate of less than 100 mg/day. The patch effectively promotes the penetration of a nucleoside antiviral agent into the blood circulation and avoids enzymolysis in the gastrointestinal tract and the first pass effect of the liver and reduces side effect of drugs, thereby inhibiting the replication of target viruses and reducing viral DNA level in the serum. A method for producing the patch is also provided. The raw materials involved in the invention are easily purchased from the market at a low cost.

### CROSS-REFERENCE TO RELATED APPLICATIONS

This application is a continuation of International Patent Application No. PCT/CN2010/077636 with an international filing date of Oct. 12, 2010, designating the United States, now pending. The contents of all of the aforementioned applications, including any intervening amendments thereto, are incorporated herein by reference.

### BACKGROUND OF THE INVENTION

1. Field of the Invention

The invention relates to an antiviral transdermal patch and a method for producing the same.

2. Description of the Related Art

Conventional antiviral drugs for treatment of viral diseases, for example, hepatitis B, AIDS, influenza virus, and herpes, include entecavir, adefovir, dipivoxil, lamivudine, acyclovir, ribavirin, and zidovudine.

Conventional dosage forms of the nucleoside antiviral drugs include oral formulations or injections. The administration modes often cause time fluctuation of blood concentrations of active ingredients. Too low concentration of active ingredients in blood cannot exhibit desired effects. Too high concentration of active ingredients in blood, however, may be poisonous to human body. Furthermore, through the administration modes, the active ingredients are easily hydrolyzed by enzymes in the gastrointestinal tract and have to experience the first pass effect of the liver, thereby reducing the efficacy.

### SUMMARY OF THE INVENTION

In view of the above-described problems, it is one objective of the invention to provide an antiviral transdermal patch that eliminates enzymolysis of administered drugs in the gastrointestinal tract and the first pass effect of the liver and reduces side effects of administered drugs.

To achieve the above objectives, in accordance with one embodiment of the invention, there is provided an antiviral transdermal patch comprising a backing layer, a viscous polymer layer, and a protection film layer, wherein the viscous polymer layer comprises an antiviral agent, a viscous polymer, and a transdermal enhancer.

In a class of this embodiment, the antiviral agent is a nucleoside antiviral drug with a daily delivery of less than 100 mg.

In a class of this embodiment, the nucleoside antiviral drug is selected from the group consisting of entecavir, adefovir dipivoxil, lamivudine, stavudine, or a mixture thereof.

In a class of this embodiment, the content of the antiviral agent per square centimeter of the antiviral transdermal patch is between 0.1 and 50 mg, preferably between 1.5 and 40 mg, more preferably between 5 and 30 mg, particularly between 10 and 25 mg, and more particularly between 15 and 20 mg.

In a class of this embodiment, the antiviral agent accounts for between 0.1 and 15.0 wt. % of the viscous polymer layer, preferably between 0.5 and 13.0 wt. %, particularly between 1.0 and 9.0 wt. %, and more particularly between 5.0 and 7.0 wt. %.

In a class of this embodiment, the viscous polymer is selected from the group consisting of a polyacrylate pressure sensitive adhesive, polyisobutylene, polyisoprene, and a silicone copolymer.

In a class of this embodiment, the polyacrylate pressure sensitive adhesive is selected from the group consisting of polyacrylic resin II, polyacrylic resin III, polyacrylic resin IV, ammonio methacrylate copolymer I, ammonio methacrylate copolymer II, and acrylic resin-EUDRAGIT E100; the acrylic resin-EUDRAGIT E100 is a copolymer of butyl methacrylate-dimethylamino ethyl methacrylate-methyl methacrylate (1:2:1).

In a class of this embodiment, the viscous polymer accounts for between 50.0 and 96.0 wt. % of the viscous polymer layer, preferably between 65.0 and 90.0 wt. %, particularly between 75.0 and 85 wt. %, and more particularly between 78.0 and 82.0 wt. %.

In a class of this embodiment, the transdermal enhancer is selected from the group consisting of laurocapram, essential oils, dimethyl sulfoxide, thymol, eucalyptus oil, and a traditional chinese medicine comprising terpenes or phenols, or a mixture thereof.

In a class of this embodiment, the transdermal enhancer is laurocapram.

In a class of this embodiment, the transdermal enhancer is a mixture of laurocapram and essential oil with a weight ratio of 1:0.5-2.

In a class of this embodiment, the transdermal enhancer is a mixture of laurocapram and dimethyl sulfoxide with a weight ratio of 1:0.5-2.

In a class of this embodiment, the transdermal enhancer accounts for between 2.0 and 12.0 wt. % of the viscous polymer layer, preferably between 4.0 and 11.0 wt. %, particularly between 6.0 and 10 wt. %, and more particularly between 8.0 and 9.0 wt. %.

In a class of this embodiment, the viscous polymer layer further comprises an organic solvent, for example, propylene glycol or ethyl lactate.

In a class of this embodiment, an adhesive layer is disposed between the viscous polymer layer and the protection film layer.

In a class of this embodiment, a raw material of the adhesive layer is selected from a polyacrylate pressure sensitive adhesive, polyisobutylene, or a silicone copolymer.

In a class of this embodiment, the backing layer is selected from the group consisting of high-density polyethylene, low density polyethylene, polypropylene, polyvinyl chloride, an ethylene-vinyl acetate copolymer, polyester, polyvinylpyrrolidone, polyvinyl alcohol, poly urethane, aluminum foil, or a mixture thereof.

In a class of this embodiment, the backing layer is a composite film formed by mixing an aluminum foil with high-density polyethylene, low density polyethylene, polypropylene, polyvinyl chloride, ethylene-vinyl acetate copolymer, polyester, polyvinylpyrrolidone, polyvinyl alcohol, poly urethane, or a mixture thereof.

In a class of this embodiment, the backing layer is a three-layered composite film with an aluminum foil in the middle and low density polyethylene, poly urethane, or a mixture thereof at both sides.

In accordance with another embodiment of the invention, there is provided a method for producing the antiviral transdermal patch comprising the steps of a) uniformly mixing the antiviral agent, the viscous polymer, the transdermal enhancer, and a solvent to yield a mixture; b) coating the mixture to the backing layer to yield the viscous polymer layer; c) drying and cooling; and d) coating the protection film layer to the viscous polymer layer.

In another aspect, the invention provides a method for treatment of viral diseases comprising administering to a patient in need thereof the antiviral transdermal patch.

Advantages of the invention are summarized below. In the invention, the viscous polymer functions as a delayed-release framework material. The antiviral agent is dissolved in the transdermal enhancer (laurocapram or a mixture thereof) uniformly distributed in the framework in the form of liquid micro-rooms through solvent evaporation. The release rate of the antiviral agent of the patch is regulated by controlling the concentration of drugs, the density of the liquid micro-rooms, and the proportion of the transdermal enhancer.

Laurocapram or a mixture thereof functioning as the transdermal enhancer effectively promotes the penetration of the nucleoside antiviral agent into skin, and then the agents is assimilated via capillary vessels and enters the blood circulation, thereby inhibiting the replication of target viruses and reducing viral DNA level in the serum. In addition, the transdermal administration avoids the enzymolysis of gastrointestinal tract and the first pass effect of the liver and reduces side effect of drugs. The resulting antiviral transdermal patch can releases active ingredients sustainably and stably for 3-5 consecutive days. The raw materials involved in the invention are simple and available from market, with a low cost.

Pharmacology and toxicology research have shown that allergy test and skin irritation test of the patch is negative. That is to say, the patch has no irritation on skin, and no hemolysis and allergy occur. Thus, the patch is safe.

Clinical studies have shown that the patch accelerates the penetration of nucleoside antiviral agent into skin, and the efficacy can be maintained for 3-5 consecutive days. The time of administration is reduced. Thus, the patch of the invention is a safe, long-term effective and convenient transdermal formulation for treatment of viral diseases.

### DETAILED DESCRIPTION OF THE EMBODIMENTS

For further illustrating the invention, experiments detailing an antiviral transdermal patch and a method for producing the same are described below. It should be noted that the following examples are intended to describe and not to limit the invention.

### EXAMPLE 1

To a jar, 185 g of polyacrylate pressure sensitive adhesive (EUDRAGIT E100, from Germany, pre-dissolved with ethyl acetate, in which solid EUDRAGIT E100 accounts for 40 wt. %) as a viscous polymer, 0.5 g of entecavir as an antiviral agent, 9 g of laurocapram as a transdermal enhancer, a mixture of 6 g of eucalyptus oil and 8.9 g of propylene glycol, and 100 g of ethyl acetate as a solvent were added. The jar was sealed and oscillated for 15 hrs, and then stood until bubbles disappeared. The resulting mixture was coated to a backing layer comprising polythene-aluminum-polyethylene to yield a viscous polymer layer which was further dried at 40° C. for 4 min, at 60° C. for 2 min, at 90° C. for 2 min, and then cooled. A non-adhesive paper was coated on the other surface of the viscous polymer layer. The product was cut into patches with an area of 2 cm² to yield an antiviral transdermal patch I with 2.5 mg of entecavir per square centimeter. The antiviral transdermal patch I comprised 75.2 wt. % polyacrylate pressure sensitive adhesive, 0.5 wt. % entecavir, 9.1 wt. % laurocapram, 6.1 wt. % eucalyptus oil, and 9 wt. % propylene glycol. The permeation rate of the patch measured using upgraded Franz diffusion cell was 15 μg/cm²/h, and the release time of the active ingredients exceeded 72 hrs. Thus, the patch was suggested for use of up to 3 days.

### EXAMPLE 2

To a jar, 225 g of polyacrylate pressure sensitive adhesive (EUDRAGIT E100, from Germany, pre-dissolved with ethyl acetate, in which solid EUDRAGIT E100 accounts for 40 wt. %) as a viscous polymer, 10 g of adefovir dipivoxil as an antiviral agent, a mixture of 9 g of laurocapram and 3 g of thymol as a transdermal enhancer, and 150 g of dichloromethane as a solvent were added. The jar was sealed and oscillated for 15 hrs, and then stood until bubbles disappeared. The resulting mixture was coated to a backing layer comprising low-density polyethylene to yield a viscous polymer layer which was further dried at 40° C. for 4 min, at 60° C. for 2 min, at 90° C. for 2 min, and then cooled. A non-adhesive paper was coated on the other surface of the viscous polymer layer. The product was cut into patches with an area of 20 cm² (4 cm×5 cm) to yield an antiviral transdermal patch II with 35 mg of adefovir dipivoxil per square centimeter. The antiviral transdermal patch II comprised 80.4 wt. % polyacrylate pressure sensitive adhesive, 9 wt. % adefovir dipivoxil, 8 wt. % laurocapram, and 2.6 wt. % thymol. The permeation rate of the patch measured using upgraded Franz diffusion cell was 20 μg/cm²/h, and the release time of the active ingredients exceeded 72 hrs. Thus, the patch was suggested for use of up to 3 days.

### EXAMPLE 3

To a jar, 128 g of polyisobutylene as a viscous polymer, 14 g of lamivudine as an antiviral agent, a mixture of 6 g of laurocapram and 10 g of essential oil as a transdermal enhancer, 42 g of ethyl lactate, and 134 g of ethyl acetate as a solvent were added. The jar was sealed and oscillated for 15 hrs, and then stood until bubbles disappeared. The resulting mixture was coated to a backing layer comprising poly urethane to yield a viscous polymer layer which was further dried at 40° C. for 4 min, at 60° C. for 2 min, at 90° C. for 2 min, and then cooled. A non-adhesive paper was coated on the other surface of the viscous polymer layer. The product was cut into patches with an area of 200 cm² to yield an antiviral transdermal patch III with 15 mg of lamivudine per square centimeter. The antiviral transdermal patch III comprised 64 wt. % polyisobutylene, 7 wt. % lamivudine, 3 wt. % laurocapram, 5 wt. % essential oil, and 21 wt. % ethyl lactate. The permeation rate of the patch measured using upgraded Franz diffusion cell was 55 μg/cm²/h, and the release time of the active ingredients exceeded 96 hrs. Thus, the patch was suggested for use of up to 4 days.

### EXAMPLE 4

To a jar, 87.5 g of polyisoprene as a viscous polymer, 15 g of stavudine as an antiviral agent, a mixture of 6 g of laurocapram and 6 g of dimethyl sulfoxide as a transdermal enhancer, and 147 g of ethyl acetate as a solvent were added. The jar was sealed and oscillated for 15 hrs, and then stood until bubbles disappeared. The resulting mixture was coated to a backing layer comprising poly urethane to yield a viscous polymer layer which was further dried at 40° C. for 4 min, at 60° C. for 2 min, at 90° C. for 2 min, and then cooled. A non-adhesive paper was coated on the other surface of the viscous polymer layer. The product was cut into patches with an area of 100 cm² to yield an antiviral transdermal patch IV with 10 mg of stavudine per square centimeter. The antiviral transdermal patch I comprised 76.5 wt. % polyisoprene, 13.1 wt. % stavudine, 5.2 wt. % laurocapram, and 5.2 wt. % dimethyl sulfoxide. The permeation rate of the patch measured using upgraded Franz diffusion cell was 34 μg/cm²/h, and the release time of the active ingredients exceeded 72 hrs. Thus, the patch was suggested for use of up to 3 days.

### EXAMPLE 5

To a jar, 88 g of silicone copolymer as a viscous polymer, 6 g of adefovir dipivoxil as an antiviral agent, a mixture of 7 g of laurocapram and 4.4 g of menthol as a transdermal enhancer, and 147 g of ethyl acetate were added. The jar was sealed and oscillated for 15 hrs, and then stood until bubbles disappeared. The resulting mixture was coated to a backing layer comprising poly urethane to yield a viscous polymer layer which was further dried at 40° C. for 4 min, at 60° C. for 2 min, at 90° C. for 2 min, and then cooled. A non-adhesive paper was coated on the other surface of the viscous polymer layer. The product was cut into patches with an area of 30 cm² to yield an antiviral transdermal patch V with 40 mg of adefovir dipivoxil per square centimeter. The antiviral transdermal patch V comprised 83.5 wt. % silicone copolymer, 5.7 wt. % adefovir dipivoxil, 6.6 wt. % laurocapram, and 4.2 wt. % menthol. The permeation rate of the patch measured using upgraded Franz diffusion cell was 18 μg/cm²/h, and the release time of the active ingredients exceeded 72 hrs. Thus, the patch was suggested for use of up to 3 days.

### EXAMPLE 6

To a jar, 225 g of polyacrylate pressure sensitive adhesive (EUDRAGIT E100, from Germany, pre-dissolved with ethyl acetate, in which solid EUDRAGIT E100 accounts for 40 wt. %) as a viscous polymer, 0.5 g of entecavir as an antiviral agent, 3.77 g of laurocapram as a transdermal enhancer, and 147 g of ethyl acetate as a solvent were added. The jar was sealed and oscillated for 15 hrs, and then stood until bubbles disappeared. The resulting mixture was coated to a backing layer comprising poly urethane to yield a viscous polymer layer which was further dried at 40° C. for 4 min, at 60° C. for 2 min, at 90° C. for 2 min, and then cooled. A layer of polyacrylate was coated on the other surface of the viscous polymer layer, dried and cooled following the process mentioned above, and then a non-adhesive paper was coated. The product was cut into patches with an area of 4 cm² (2 cm×2 cm) to yield an antiviral transdermal patch VI with 3.25 mg of entecavir per square centimeter. The antiviral transdermal patch VI comprised 95.5 wt. % polyacrylate pressure sensitive adhesive, 0.5 wt. % entecavir, and 4 wt. % laurocapram. The permeation rate of the patch measured using upgraded Franz diffusion cell was 10 μg/cm²/h, and the release time of the active ingredients exceeded 48 hrs. Thus, the patch was suggested for use of up to 2 days.

### EXAMPLE 7

To a jar, 225 g of polyacrylate pressure sensitive adhesive (EUDRAGIT E100, from Germany, pre-dissolved with ethyl acetate, in which solid EUDRAGIT E100 accounts for 40 wt. %) as a viscous polymer, 0.2 g of entecavir as an antiviral agent, 9.8 g of laurocapram as a transdermal enhancer, 6.38 g of propylene glycol, and 160 g of ethyl acetate as a solvent were added. The jar was sealed and oscillated for 15 hrs, and then stood until bubbles disappeared. The resulting mixture was coated to a backing layer comprising polythene-aluminum-polyethylene to yield a viscous polymer layer which was further dried at 40° C. for 4 min, at 60° C. for 2 min, at 90° C. for 2 min, and then cooled. A layer of silicone copolymer was coated on the other surface of the viscous polymer layer, dried and cooled following the process mentioned above, and then a non-adhesive paper was coated. The product was cut into patches with an area of 4 cm² (2 cm×2 cm) to yield an antiviral transdermal patch VII with 2.2 mg of entecavir per square centimeter. The antiviral transdermal patch VII comprised 84.6 wt. % polyacrylate pressure sensitive adhesive, 0.19 wt. % entecavir, 9.21 wt. % laurocapram, and 6 wt. % propylene glycol. The permeation rate of the patch measured using upgraded Franz diffusion cell was 12 μg/cm²/h, and the release time of the active ingredients exceeded 72 hrs. Thus, the patch was suggested for use of up to days.

### EXAMPLE 8

To ajar, 170 g of polyacrylate pressure sensitive adhesive (EUDRAGIT E100, from Germany, pre-dissolved with ethyl acetate, in which solid EUDRAGIT E100 accounts for 40 wt. %) as a viscous polymer, 0.8 g of entecavir as an antiviral agent, a mixture of 4 g of laurocapram and 5.4 g of eucalyptus oil as a transdermal enhancer, 5 g of propylene glycol, and 100 g of ethyl acetate were added. The jar was sealed and oscillated for 15 hrs, and then stood until bubbles disappeared. The resulting mixture was coated to a backing layer comprising poly urethane to yield a viscous polymer layer which was further dried at 40° C. for 4 min, at 60° C. for 2 min, at 90° C. for 2 min, and then cooled. A non-adhesive paper was coated on the other surface of the viscous polymer layer. The product was cut into patches with an area of 2 cm² (1 cm×2 cm) to yield an antiviral transdermal patch VIII with 6 mg of entecavir per square centimeter. The antiviral transdermal patch VIII comprised 82 wt. % polyacrylate pressure sensitive adhesive, 1 wt. % entecavir, 4.5 wt. % laurocapram, 6.5 wt. % eucalyptus oil, and 6 wt. % propylene glycol. The permeation rate of the patch measured using upgraded Franz diffusion cell was 15 μg/cm²/h, and the release time of the active ingredients exceeded 72 hrs. Thus, the patch was suggested for use of up to 3 days.

### EXAMPLE 9

To a jar, 200 g of polyacrylate pressure sensitive adhesive (EUDRAGIT E100, from Germany, pre-dissolved with ethyl acetate, in which solid EUDRAGIT E100 accounts for 40 wt. %) as a viscous polymer, 1 g of entecavir as an antiviral agent, a mixture of 8 g of laurocapram and 8.5 g of eucalyptus oil as a transdermal enhancer, and 100 g of ethyl acetate as a solvent were added. The jar was sealed and oscillated for 15 hrs, and then stood until bubbles disappeared. The resulting mixture was coated to a backing layer comprising polythene-aluminum-polyethylene to yield a viscous polymer layer which was further dried at 40° C. for 4 min, at 60° C. for 2 min, at 90° C. for 2 min, and then cooled. A non-adhesive paper was coated on the other surface of the viscous polymer layer. The product was cut into patches with an area of 2 cm² (1 cm×2 cm) to yield an antiviral transdermal patch IX with 7 mg of entecavir per square centimeter. The antiviral transdermal patch IX comprised 82 wt. % polyacrylate pressure sensitive adhesive, 1.5 wt. % entecavir, 8 wt. % laurocapram, and 8.5 wt. % eucalyptus oil. The permeation rate of the patch measured using upgraded Franz diffusion cell was 12 μg/cm²/h, and the release time of the active ingredients exceeded 96 hrs. Thus, the patch was suggested for use of up to 4 days.

### EXAMPLE 10

To a jar, 150 g of polyacrylate pressure sensitive adhesive (EUDRAGIT E100, from Germany, pre-dissolved with ethyl acetate, in which solid EUDRAGIT E100 accounts for 40 wt. %) as a viscous polymer, 0.5 g of entecavir as an antiviral agent, a mixture of 10 g of laurocapram and 10 g of eucalyptus oil as a transdermal enhancer, 9.5 g of propylene glycol, and 100 g of ethyl acetate were added. The jar was sealed and oscillated for 15 hrs, and then stood until bubbles disappeared. The resulting mixture was coated to a backing layer comprising low density polythene to yield a viscous polymer layer which was further dried at 40° C. for 4 min, at 60° C. for 2 min, at 90° C. for 2 min, and then cooled. A layer of polyisobutylene was coated on the other surface of the viscous polymer layer, dried and cooled following the process mentioned above, and then a non-adhesive paper was coated. The product was cut into patches with an area of 1 cm² (1 cm×1 cm) to yield an antiviral transdermal patch X with 0.8 mg of entecavir per square centimeter. The antiviral transdermal patch X comprised 66.4 wt. % polyacrylate pressure sensitive adhesive, 0.6 wt. % entecavir, 11 wt. % laurocapram, 11 wt. % eucalyptus oil, and 11 wt. % propylene glycol. The permeation rate of the patch measured using upgraded Franz diffusion cell was 22 μg/cm²/h, and the release time of the active ingredients exceeded 72 hrs. Thus, the patch was suggested for use of up to 3 days.

### EXAMPLE 11

### Irritation Test of Entecavir Transdermal Patch

Drug to be tested: entecavir

Patch to be tested: a patch prepared by Example 7

Patch for control groups: a patch prepared by Example 7 but no entecavir and transdermal enhancer added.

Method: 40 healthy guinea pigs (180 g±20 g), half male and half female, were collected and carried out with left/right self comparison experiments. At 24 hrs prior to the experiments, the hair of drug administration area (on the back) of guinea pigs was removed, with an area of 3 cm×3 cm at both sides. The patch to be tested was pasted on one side and the patch for the control pasted on the other side. The patches were pasted for 8 hrs per day in 3 consecutive weeks. When the patches were removed, the drug administration area was cleaned with warm water or a non-irritating solvent. After one hour of patch removal as well as prior to next pasting, the guinea pigs were examined whether there were erythema, edema, pigmentation, blood spots, rough skin, or thin skin in the drug administration area. After 30-60 min, 24 hrs, 48 hrs, and 72 hrs of the final patch removal, the guinea pigs were examined with naked eyes whether there were erythema and edema in the drug administration area.

Results and conclusions: During the experiments, the appearance, behavior, feeding, and excretion of the guinea pigs were as normal as before, no abnormal pruritus occurred. The body weight of all guinea pigs increased, and no erythema, edema, pigmentation, blood spots, rough skin, or thin skin observed in the drug administration area. No guinea pig died. Thus, the patch had no obvious irritation.

### EXAMPLE 12

### Irritation Test of Lamivudine Transdermal Patch

Drug to be tested: lamivudine

Patch to be tested: a patch prepared by Example 3

Patch for control groups: a patch prepared by Example 3 but no lamivudine and transdermal enhancer added.

Method: 30 healthy rabbits, half male and half female, were collected and carried out with left/right self comparison experiments. At 24 hrs prior to the experiments, the hair of drug administration area (on the back) of the rabbits was removed, with an area of 4 cm×4 cm at both sides. The patch to be tested was pasted on one side and the patch for the control pasted on the other side. The patches were pasted for 8 hrs per day in 3 consecutive weeks. When the patches were removed, the drug administration area was cleaned with warm water or a non-irritating solvent. After one hour of patch removal as well as prior to next pasting, the rabbits were examined whether there were erythema, edema, pigmentation, blood spots, rough skin, or thin skin in the drug administration area. After 30-60 min, 24 hrs, 48 hrs, and 72 hrs of the final patch removal, the rabbits were examined with naked eyes whether there were erythema and edema in the drug administration area.

Results and conclusions: During the experiments, the appearance, behavior, feeding, and excretion of the rabbits were as normal as before, no abnormal pruritus occurred. The body weight of all rabbits increased, and no erythema, edema, pigmentation, blood spots, rough skin, or thin skin observed in the drug administration area. No rabbit died. Thus, the patch had no obvious irritation.

### EXAMPLE 13

### Irritation Test of Stavudine Transdermal Patch

Drug to be tested: stavudine

Patch to be tested: a patch prepared by Example 4

Patch for control groups: a patch prepared by Example 4 but no stavudine and transdermal enhancer added.

Method: 30 healthy rabbits, half male and half female, were collected and carried out with left/right self comparison experiments. At 24 hrs prior to the experiments, the hair of drug administration area (on the back) of the rabbits was removed, with an area of 4 cm×4 cm at both sides. The patch to be tested was pasted on one side and the patch for the control pasted on the other side. The patches were pasted for 8 hrs per day in 3 consecutive weeks. When the patches were removed, the drug administration area was cleaned with warm water or a non-irritating solvent. After one hour of patch removal as well as prior to next pasting, the rabbits were examined whether there were erythema, edema, pigmentation, blood spots, rough skin, or thin skin in the drug administration area. After 30-60 min, 24 hrs, 48 hrs, and 72 hrs of the final patch removal, the rabbits were examined with naked eyes whether there were erythema and edema in the drug administration area.

Results and conclusions: During the experiments, the appearance, behavior, feeding, and excretion of the rabbits were as normal as before, no abnormal pruritus occurred. The body weight of all rabbits increased, and no erythema, edema, pigmentation, blood spots, rough skin, or thin skin observed in the drug administration area. No rabbit died. Thus, the patch had no obvious irritation.

### EXAMPLE 14

### Allergy Test of Entecavir Transdermal Patch

Drug to be tested: entecavir

Patch to be tested: a first patch whose entecavir concentration was four times that of a patch prepared by Example 7 as a sensitizer; a second patch whose entecavir concentration was eight times that of a patch prepared by Example 7 as an activator.

Negative control group: a patch prepared by Example 7 but no entecavir and transdermal enhancer added.

Positive control group: 1% mercapto thiazole as a sensitizer and 2% mercapto thiazole as an activator.

Method: 30 healthy adult guinea pigs, either male or female, were divided into an experimental group (20), a positive control group (5), and a negative control group (5). At 24 hrs prior to the experiments, the hair of drug administration area (on the back) of the guinea pigs was removed, with an area of 4 cm×4 cm at both sides. Buehler test (BT) was carried out as follows. The guinea pigs were administered with a sensitizer for 10-14 days to induce immunoreactions, and then an activator was administered. Observe whether there were allergic reactions. The skin response and response degree thereof at the induction period and attack period were compared, and the results were compared with those of the negative control group.

During the Buehler test (BT), entecavir transdermal patches were pasted at the 0, 6^{th}, and 13^{th} days on one side of the abdomen for induction, and at the 27^{th} day pasted on the other side for 6 hrs for activation. At one hour and 24 hrs after sensitization and 24 and 48 hrs after activation, observe whether there were erythema, edema, and other abnormal reactions, and evaluate the erythema and edema. The results were listed as Table 1.

TABLE 1
Skin allergy rate (%)

| Groups | 0 h | 24 h | 48 h | 72 h | Evaluation on sensitization |
|---|---|---|---|---|---|
| Mercapto thiazole | 60 | 80 | 90 | 80 | Allergenic |
| Negative control | 0 | 0 | 0 | 0 | Nonallergenic |
| Entecavir | 0 | 0 | 0 | 0 | Nonallergenic |

The results showed that, the patch of the invention had no obvious allergenicity.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. An antiviral transdermal patch comprising a backing layer, a viscous polymer layer, and a protection film layer, wherein said viscous polymer layer comprises an antiviral agent, a viscous polymer, and a transdermal enhancer.

2. The patch of claim 1, wherein said antiviral agent is an antiviral nucleoside drug with a daily administration rate of less than 100 mg/day.

3. The patch of claim 2, wherein said antiviral nucleoside drug is selected from the group consisting of entecavir, adefovir dipivoxil, lamivudine, stavudine, or a mixture thereof.

4. The patch of claim 1, wherein the content of said antiviral agent per square centimeter of said antiviral transdermal patch is between 0.1 and 50 mg.

5. The patch of claim 1, wherein said antiviral agent accounts for between 0.1 and 15.0 wt. % of said viscous polymer layer.

6. The patch of claim 1, wherein said viscous polymer is selected from the group consisting of a polyacrylate pressure sensitive adhesive, polyisobutylene, polyisoprene, and a silicone copolymer.

7. The patch of claim 6, wherein said polyacrylate pressure sensitive adhesive is selected from the group consisting of polyacrylic resin II, polyacrylic resin III, polyacrylic resin IV, ammonio methacrylate copolymer I, and ammonio methacrylate copolymer II.

8. The patch of claim 1, wherein said viscous polymer accounts for between 50.0 and 96.0 wt. % of said viscous polymer layer.

9. The patch of claim 1, wherein said transdermal enhancer is selected from the group consisting of laurocapram, essential oils, dimethyl sulfoxide, thymol, eucalyptus oil, a traditional chinese medicine comprising terpenes or phenols, or a mixture thereof.

10. The patch of claim 9, wherein said transdermal enhancer is a mixture of laurocapram and essential oil with a weight ratio of 1:0.5-2

11. The patch of claim 9, wherein said transdermal enhancer is a mixture of laurocapram and dimethyl sulfoxide with a weight ratio of 1:0.5-2.

12. The patch of claim 1, wherein said transdermal enhancer accounts for between 2.0 and 12.0 wt. % of said viscous polymer layer.

13. The patch of claim 1, wherein said viscous polymer layer further comprises an organic solvent.

14. The patch of claim 1, wherein an adhesive layer is disposed between said viscous polymer layer and said protection film layer.

15. The patch of claim 14, wherein a raw material of said adhesive layer is selected from a polyacrylate pressure sensitive adhesive, polyisobutylene, or a silicone copolymer.

16. The patch of claim 1, wherein said backing layer is selected from the group consisting of high-density polyethylene, low density polyethylene, polypropylene, polyvinyl chloride, ethylene-vinyl acetate copolymer, polyester, polyvinylpyrrolidone, polyvinyl alcohol, poly urethane, aluminum foil, or a mixture thereof.

17. The patch of claim 16, wherein said backing layer is a composite film formed by mixing an aluminum foil with high-density polyethylene, low density polyethylene, polypropylene, polyvinyl chloride, ethylene-vinyl acetate copolymer, polyester, polyvinylpyrrolidone, polyvinyl alcohol, or poly urethane.

18. The patch of claim 16, wherein said backing layer is a three-layered composite film with an aluminum foil in the middle and low density polyethylene, poly urethane, or a mixture thereof at both sides.

19. A method for producing the patch of claim 1, comprising the steps of a) uniformly mixing said antiviral agent, said viscous polymer, said transdermal enhancer, and a solvent to yield a mixture; b) coating said mixture to said backing layer to yield said viscous polymer layer; c) drying and cooling; and d) coating said protection film layer to said viscous polymer layer.

20. A method for treatment of viral diseases comprising administering to a patient in need thereof the antiviral transdermal patch of claim 1.
